(19) United States
(12) Patent Application Publication
(10) Pub. No.: US 2026/0001874 A1
(43) Pub. Date: Jan. 1, 2026

(54) DIHYDROQUINAZOLINONES EXHIBITING IMPROVED PROTECTIVE ACTIVITY AGAINST INTRACELLULARLY ACTING TOXINS AND INTRACELLULAR VIRUSES AND BACTERIA

(71) Applicant: COMMISSARIAT À L'ENERGIE ATOMIQUE ET AUX ENERGIES ALTERNATIVES, Paris (FR)

(72) Inventors: Daniel GILLET, GIF-SUR-YVETTE (FR); Jean-Christophe CINTRAT, GIF-SUR-YVETTE (FR); Julien BARBIER, GIF-SUR-YVETTE (FR); Lucie CARAMELLE, GIF-SUR-YVETTE (FR)

(73) Assignee: COMMISSARIAT À L'ENERGIE ATOMIQUE ET AUX ENERGIES ALTERNATIVES, Paris (FR)

(21) Appl. No.: 18/854,174

(22) PCT Filed: Mar. 28, 2023
(86) PCT No.: PCT/EP2023/058044

§ 371 Date: Oct. 4, 2024

(30) Foreign Application Priority Data
Apr. 8, 2022 (FR) FR2203227

(51) Int. Cl. C07D 417/14; A61K 31/517; A61P 31/04; A61P 31/20; A61P 39/00

(52) U.S. Cl. CPC C07D 417/14; A61K 31/517; A61P 31/04; A61P 31/20; A61P 39/00

(57) ABSTRACT

A compound of general formula (I) and also the stereoisomeric forms, mixtures of stereoisomeric forms or pharmaceutically acceptable salts thereof, and also to the use thereof for the prevention and/or treatment of disorders caused by intracellularly acting toxins using retrograde transport, or by viruses or bacteria using retrograde and/or syntaxin 5-dependent transport to infect cells.

The present invention relates to a new class of 2,3-dihydroquinazolin-4(1H)-one compounds and to their use as inhibitors of the toxic effects of toxins with intracellular activity, such as, for example, ricin, abrin and Shiga toxins, which utilize retrograde transport for intoxicating the cells, or of viruses or bacteria which utilize retrograde and/or syntaxin 5-dependent transport for infecting the cells, particularly viruses or bacteria which enter the cells by endocytosis, or of intracellular parasites.

### TECHNICAL FIELD

Toxins with intracellular activity which utilize retrograde transport represent a major risk in public health and some of them are associated with more than a million deaths annually worldwide. These toxins are, particularly, ricin (produced in the seeds of the plant Ricinus communis), abrin (produced in the seeds of the plant Abrus precatorius), Shiga toxin and Shiga-like toxins (Stxs) produced by Shigella dysenteriae (Stx) and E. coli (Stx1 and Stx2), the pertussis toxin (Bordetella pertussis, the agent of whooping cough), subtilase cytotoxin and heat-labile enterotoxin (E. coli).

Ricin is a 66 kDa glycoprotein composed of two polypeptide chains linked by a disulfide bridge. The B chain (RTB, lectin of 262 residues) enables the toxin to bind to the glycolipids and glycoproteins of the cell membranes and to obtain entry into the cell. The A chain (RTA, 267 amino acids) ensures the N-glycosidase enzymatic function of the ricin, catalyzes the elimination of adenine 4324 from the 28S RNA of the targeted cells, and causes protein synthesis to come to a halt.

Abrin is an extremely toxic toxalbumin present in the seeds of Abrus precatorius and has a structure and a mechanism of action similar to those of ricin.

Shiga toxins include the Shiga toxin (Stx) produced by Shigella dysenteriae and the Shiga-like toxins 1 (Stx1) and 2 (Stx2) produced by enterohemorragic strains of E. coli. The toxins Stx and Stx1 are 99% identical, whereas Stx1 and Stx2 share only 56% identity in their amino acid sequence. Subunit A of the toxin (StxA) carries the same enzymatic activity as ricin and in exactly the same way targets the 28S RNA. Subunit B (StxB), which is pentameric, allows the toxin to bind to the cell via its interaction with the globo-triaosylceramide Gb3, thereby ensuring its internalization and its intracellular routing. After binding to its membrane receptors, ricin is internalized in these cells by multiple pathways of endocytosis to reach the trans-Golgi network, where it is conveyed to the endoplasmic reticulum (ER) by retrograde transport (Johannes, L. et al., Cell 2008, 135, 1175-87). Abrin acts by penetrating the cell, binding to the carbohydrate chains of the glycoproteins and glycolipids at the cell surface before being internalized in the interior of said cell. This process, which is not well known, is similar in terms of structure and mechanism of action to that of ricin. Shiga toxins are for their part internalized by a unique pathway of endocytosis and likewise reach the Golgi apparatus and then the endoplasmic reticulum (ibid.). The toxins are then partially unfolded and the chain/subunit A is translocated into the cytosol (Lord, J. M. et al., Biochemical Society Transactions 2003, 31, 1260). The final step in the action of these toxins therefore takes place in the cytoplasm of the cells; the toxins affix to the ribosomes with a high efficacy and cleave adenine 4324 from the 28S RNA of subunit 60S of the ribosome. This depurination of the 28S RNA causes protein synthesis to come to a halt and leads to cell death.

To counter the threat posed by these toxins, a number of types of antitoxins have been developed: neutralizing antibodies, inhibitors of enzymatic activity (small molecules, substrate analogues), soluble receptor mimics, and chemical compounds which act on the cells targeted by the toxin.

Recent years have seen acceleration in the search for new molecules aiming to block the intracellular routing of the toxins with intracellular activity. The primary advantage of this type of molecules is their broad-spectrum activity, as these molecules are able to provide cells with effective protection against the various toxins which utilize the retrograde pathway.

Poxviruses are DNA viruses allied to the family Poxviridae, and are infectious for animals and responsible for diseases such as monkeypox, or “simian smallpox”, smallpox, vaccinia, cowpox infection, or “cow smallpox”, camelpox infection, which affects camelids (camels, dromedaries, llamas), or else molluscum contagiosum. Poxviroses with an animal reservoir, more particularly monkeypox and camelpox, are emergent diseases with a risk of spread owing to the increase in international transport, to the fashion for companion animals, particularly exotic animals, to the proximity of people to their draft animals, and to the absence of smallpox vaccine protection. There is an anti-smallpox molecule on the market: ST246-tecovirimat or TPOXX®. However, the monotherapy with this molecule gives rise to resistant viral strains and there is a consensus on the need to have several anti-poxvirus medicaments, particularly in a OneHealth approach.

There consequently remains a great need to provide effective treatments against this family of viruses.

The present invention aims precisely to provide molecules enabling protection for cells against toxins with intracellular activity, against viruses or bacteria, and intracellular parasites.

According to a first embodiment, the present invention relates to a compound of general formula (I′):

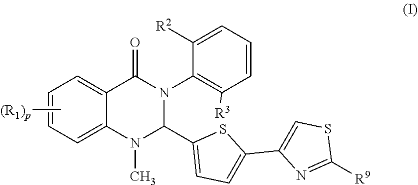

in which
p is 1, 2 or 3;
R¹ represents at each occurrence, independently, a hydrogen atom, a halogen atom, an alkoxy radical of 1 to 3 carbon atoms, particularly a methoxy group, —NO₂, or —NH₂;
R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, or a halogen atom;
R⁴, R⁵ and R⁶ represent independently of one another a group of formula (II) -L-(X)i-(PEG)-(Y)j-Z, in which:
   i and j represent independently of one another 0 or 1;
   L represents —C(═O)— or —C(═O)—(CH₂)k-C(═O)—, with k being 1, 2 or 3, more particularly 2;
R⁷ and R⁸ represent, independently of one another, a linear or branched C₁ to C₃ alkyl;
X and Y represent independently of one another a poly(lactic acid) or a poly(lactic acid-co-glycolic acid);
PEG represents a poly(ethylene glycol);
Z represents a group chosen from H, a C₁ to C₃ alkyl, —OH, a C₁ to C₃ O-alkyl, or -L-Re, in which L is defined as above and Re is a residue of formula (I) connected to said group -L-(X)i-(PEG)-(Y)j-defined above by way of its group R² or R³, said group R² or R³ being —OH, —NH₂ or —SO₂—NH₂;
R⁹ represents —CH₃ or —CH₂—OH, and more particularly represents CH₃,
and also the stereoisomeric forms, the mixtures of stereoisomeric forms, or the pharmaceutically acceptable salts thereof.

According to another embodiment, the present invention relates to a compound of general formula (I′):

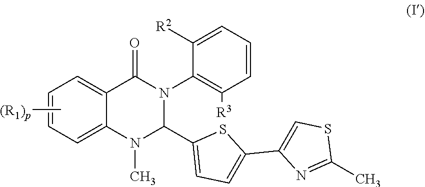

in which
p is 1, 2 or 3;
R¹ represents at each occurrence, independently, a hydrogen atom, a halogen atom, an alkoxy radical of 1 to 3 carbon atoms, particularly a methoxy group, —NO₂, or —NH₂;
R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, or a halogen atom;
R⁴, R⁵ and R⁶ represent independently of one another a group of formula (II) -L-(X)i-(PEG)-(Y)j-Z, in which:
   i and j represent independently of one another 0 or 1;
   L represents —C(═O)— or —C(═O)—(CH₂) k-C(═O)—, with k being 1, 2 or 3, more particularly 2;
R⁷ and R⁸ represent, independently of one another, a linear or branched C₁ to C₃ alkyl;
X and Y represent independently of one another a poly(lactic acid) or a poly(lactic acid-co-glycolic acid);
PEG represents a poly(ethylene glycol);
Z represents a group chosen from H, a C₁ to C₃ alkyl, —OH, a C₁ to C₃ O-alkyl, or -L-Re, in which L is defined as above and Re is a residue of formula (I) connected to said group -L-(X)i-(PEG)-(Y)j-defined above by way of its group R² or R³, said group R² or R³ being —OH, —NH₂ or —SO₂—NH₂;
and also the stereoisomeric forms, the mixtures of stereoisomeric forms, or the pharmaceutically acceptable salts thereof.

To particular surprise, this new family of molecules exhibits a biological activity which is as effective as or even superior to that of compounds from the family of molecules described in international patent application WO 2020/109510 A1. Very similar structurally, these molecules differ from those of the present invention in the presence of one or two ortho and meta substitutions in the phenyl ring.

Moreover, the molecules of the present invention exhibit a better calculated solubility (see column “log S” in table 3 hereinafter), which is manifested, for certain molecules only, unexpectedly, in a better bioavailability relative to the molecules known in the art, and particularly relative to the compounds A to D presented in tables 2 and 3 hereinafter. These properties are accrued particularly when the molecules are administered in a vehicle solution as described hereinafter. Indeed, the poor solubility of the compound Retro-2.1 and its analogues in the “conventional” administration vehicles poses a major problem to formulators, more particularly when the aim is to produce pharmaceutical compositions at scale.

According to a second aspect, the invention relates to a compound of general formula (I) as defined above, for use thereof for the prevention and/or the treatment of disorders induced by toxins having an intracellular mode of action which utilize retrograde transport, or by viruses or bacteria which utilize retrograde and/or syntaxin 5-dependent transport for infecting the cells, particularly viruses or bacteria which enter the cells by endocytosis, or by intracellular parasites.

The invention also relates to a pharmaceutical composition or a medicament comprising at least one compound of general formula (I) as defined above as active principle, and a pharmaceutically acceptable vehicle, said pharmaceutical composition or said medicament being suitable particularly for administration by aerial, oral, parenteral, local, intramuscular or subcutaneous routes.

### BRIEF DESCRIPTION OF THE DRAWINGS

FIG. 1 represents the plasma concentration (in nM) of compound 1 (white circle), of compound B (white square), of compound C (white triangle) and of compound E (white diamond) as a function of time (in hours) administered in the female BALB/c mouse orally at a concentration of 33 mg/kg in a Lipiodol®/castor oil mixture in proportions by mass of 30%/70%.

FIG. 2 represents the plasma concentration (in nM) of compound 1 (white circle), of compound B (white square), of compound C (white triangle) and of compound E (white diamond) as a function of time (in hours) administered in the female BALB/c mouse subcutaneously at a concentration of 33 mg/kg in a Lipiodol®/castor oil mixture in proportions by mass of 30%/70%.

FIG. 3 represents the plasma concentration (in nM) of compound 1 in formulation B (white circle), of compound 1 in formulation C (black circle), of compound B in formulation B (white square) and of compound B in formulation C (black square) as a function of time (in hours) administered subcutaneously in the female BALB/c mouse at a concentration of 33 mg/kg.

FIG. 4 represents a simulation of the plasma concentration (in nM) of compound B (left-hand graph) or of compound 1 (right-hand graph) as a function of time (in hours) administered twice daily subcutaneously in the female BALB/c mouse at a concentration of 33 mg/kg in a formulation C.

FIG. 5 represents the residual percentage (in %) of the plasma concentration of compound 1 (curve 1), of compound A (curve 2), of compound B (curve 5), of compound C (curve 4) and of compound E (curve 3) as a function of time (in hours) incubated for 45 minutes with murine (A) or human (B) hepatic microsomes in the presence of NADPH as a function of time (in minutes).

In FIG. 5A, the curves at 30 hours are, from top to bottom, those of compound 1, of compound A, of compound E, of compound C and of compound B.

In FIG. 5B, the curves at 30 hours are, from top to bottom, those of compound E, of compound 1, of compound B and of compound C.

FIG. 6 represents a simulation of the plasma concentration (in μM) of (S)-compound 1 as a function of time (in hours) administered twice daily subcutaneously (upper graph) or once daily subcutaneously (lower graph) in the female BALB/c mouse at a concentration of 33 mg/kg.

### DISCLOSURE OF THE INVENTION

In the course of its research into compounds which block retrograde transport, and more particularly of studies into compounds which are more advantageous than those of the abovementioned works, the Applicant has identified a new family of compounds which are derivatives of 2,3-dihydroquinazolin-4 (1H)-one, bearing a phenyl specifically substituted in diortho (ortho and ortho) positions, which unexpectedly exhibit a biological activity greater than that of Retro-2.2, the most active of the molecules already described, and hence a marked interest for the prevention and/or the treatment of intoxications with at least one toxin with an intracellular mode of action that utilizes retrograde transport for infecting eukaryotic cells in mammals, but also for the prevention and/or the treatment of infections with viruses or bacteria which utilize retrograde and/or syntaxin 5-dependent transport for infecting the cells, particularly the viruses or bacteria which enter the cells by endocytosis, or with intracellular parasites.

Accordingly, the present invention relates to a compound of general formula (I):

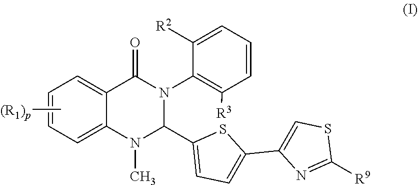

in which
p is 1, 2 or 3;
R¹ represents at each occurrence, independently, a hydrogen atom, a halogen atom, an alkoxy radical of 1 to 3 carbon atoms, particularly a methoxy group, —NO₂, or —NH₂;
R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, or a halogen atom;
R⁴, R⁵ and R⁶ represent independently of one another a group of formula (II) -L-(X)i-(PEG)-(Y)j-Z, in which:
   i and j represent independently of one another 0 or 1;
   L represents —C(═O)— or —C(═O)—(CH₂)k-C(═O)—, with k being 1, 2 or 3, more particularly 2;
R⁷ and R⁸ represent, independently of one another, a linear or branched C₁ to C₃ alkyl;
X and Y represent independently of one another a poly(lactic acid) or a poly(lactic acid-co-glycolic acid);
PEG represents a poly(ethylene glycol);
Z represents a group chosen from H, a C₁ to C₃ alkyl, —OH, a C₁ to C₃ O-alkyl, or -L-Re, in which L is defined as above and Re is a residue of formula (I) connected to said group -L-(X)i-(PEG)-(Y)j-defined above by way of its group R² or R³, said group R² or R³ being —OH, —NH₂ or —SO₂—NH₂;
R⁹ represents —CH₃ or —CH₂—OH, and more particularly represents CH₃, and also the stereoisomeric forms, the mixtures of stereoisomeric forms, or the pharmaceutically acceptable salts thereof.

According to one embodiment, R⁹ represents —CH₃.

According to one embodiment, p is 1.

According to one embodiment, R¹ represents a halogen atom, more particularly a fluorine atom.

According to one embodiment, the invention relates to a compound of general formula (I) wherein R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, and more particularly R³ represents an —OH group, with R⁴, R⁵, R⁶, R⁷ and R⁸ being defined as above.

According to one embodiment, the compound of general formula (I) according to the invention is of formula (Ia) below:

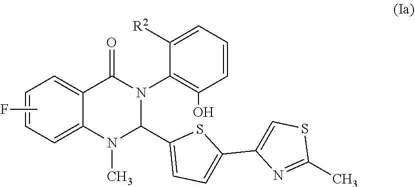

in which R² is as defined above.

According to one particular embodiment, the compound of general formula (I) according to the invention is of formula (Ib) below:

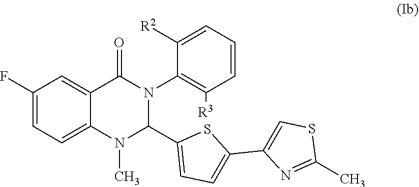

wherein R² and R³ are chosen from the following combinations: R² is an —OH group and R³ is an —NH₂ group, R² is an —OH group and R³ is an —SO₂NH₂ group, R² is an —OH group and R³ is an —SH group, R² is an —OH group and R³ is an —SO₃H group, R² is an —OH group and R³ is an —SMe group, R² is an —OH group and R³ is an —OMe group, R² is an —OH group and R³ is a halogen atom and more particularly is a fluorine atom, R² is an —NH₂ group and R³ is an —NH₂ group, R² is an —NH₂ group and R³ is an —SO₂NH₂ group, R² is an —NH₂ group and R³ is an —SH group, R² is an —NH₂ group and R³ is an —SO₃H group, R² is an —NH₂ group and R³ is an —SMe group, R² is an —NH₂ group and R³ is an —OMe group, R² is an —NH₂ group and R³ is a halogen atom, and more particularly is a fluorine atom, R² is an —SH group and R³ is an —SH group, R² is an —SH group and R³ is an —SO₂NH₂ group, R² is an —SH group and R³ is an —SO₃H group, R² is an —SH group and R³ is an —SMe group, R² is an —SH group and R³ is an —OMe group, R² is an —SH group and R³ is a halogen atom, and more particularly is a fluorine atom, R² is an —OMe group and R³ is an —OMe group, R² is an —OMe group and R³ is an —SMe group, R² is an —OMe group and R³ is a halogen atom, and more particularly is a fluorine atom, R² is an —SMe group and R³ is an —SMe group, R² is an —SMe group and R³ is a halogen atom, and more particularly is a fluorine atom, and R² is halogen atom and more particularly is a fluorine atom and R³ is a halogen atom and more particularly is a fluorine atom, where Me is a methyl.

According to one particular embodiment, the compound has the formula below:

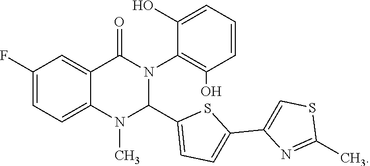 [SMILES: Cc1nc(-c2ccc(C3N(C)c4ccc(F)cc4C(=O)N3c3c(O)cccc3O)s2)cs1]

In the patent application, this compound will bear the name “compound 1”.

According to one particular embodiment, the compound has the formula below:

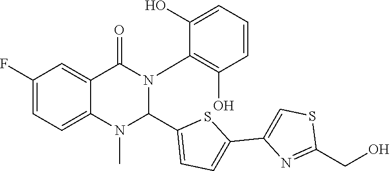 [SMILES: CN1c2ccc(F)cc2C(=O)N(c2c(O)cccc2O)C1c1ccc(-c2csc(CO)n2)s1]

This compound is a metabolite of compound 1. A “metabolite” is understood to be a transformed form of a compound, following the administration of said compound in vivo or in vitro. For example, this transformation may consist of hydroxylation of the compound.

With regard to the group of formula (II) -L-(X)ᵢ—(PEG)-(Y)ⱼ—Z, L represents particularly —C(═O)— when R² or R³ represents —NHR⁵ or —SO₂—NH—R⁶, and L represents particularly —C(═O)—(CH₂)ₖ—C(═O)— when R² or R³ represents —OR⁴.

According to one embodiment, the group of formula (II) -L-(X)ᵢ—(PEG)-(Y)ⱼ—Z is chosen from -L-PEG, -L-PEG-PLA and -L-PLGA-PEG-PLGA, with L representing particularly —C(═O)— when R² or R³ represents —NHR⁵ or —SO₂—NH—R⁶, or particularly —C(═O)—(CH₂)ₖ—C(═O)— when R² or R³ represents —OR⁴.

According to one embodiment, the group of formula (II) -L-(X)ᵢ—(PEG)-(Y)ⱼ—Z is chosen from —C(═O)—(CH₂—CH₂—O)ₘ—CH₃, particularly when R² or R³ represents —NHR⁵ or —SO₂—NH—R⁶, and —C(═O)—(CH₂)ₖ—C(═O)—(O—CH₂—CH₂)ₘ—OCH₃, particularly when R² or R³ represents —OR⁴, with m being from 1 to 500, m being more particularly from 30 to 60, more particularly still 45.

According to one embodiment, the compound of general formula (I) is of formula (III) below:

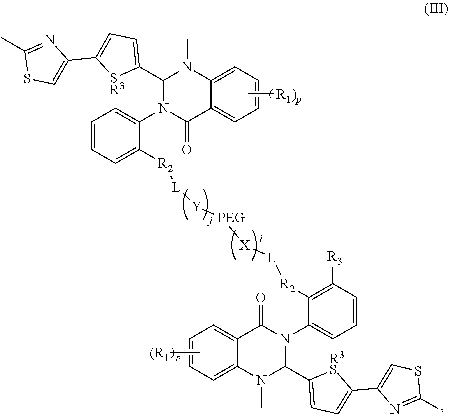

with the groups mentioned in the formula (III) being as defined earlier on above. According to one embodiment, the compound of general formula (I) according to the invention is chosen from:

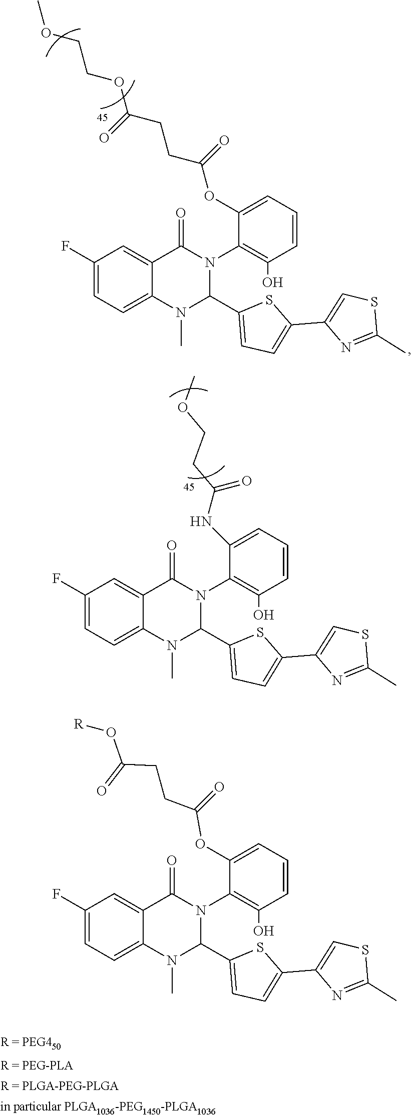

and (Compound 1)-PLGA1036-PEG1450-PLGA1036-(Compound 1) or (Compound 1)-C(═O)—(CH₂)₂—C(═O)—PLGA1036-PEG1450-PLGA1036-C(═O)—(CH₂)₂—C(═O)-(Compound 1), more particularly of formula below:

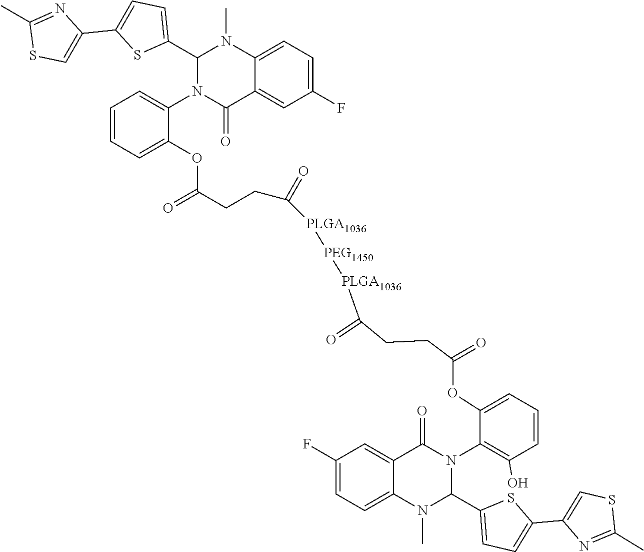

The compounds of formula (I) may be in the form of an R enantiomer, in the form of an S enantiomer, or in the form of a racemic mixture. According to one particular embodiment, the compounds of formula (I) may be in the form of an S enantiomer.

Some compounds of general formula (I) constitute prodrugs. Illustratively, mention may be made of the compounds of general formula (I) for which R⁴, R⁵ and R⁶ represent independently of one another a group of formula (II) -L-(X)i-(PEG)-(Y)j-Z, in which:
i and j represent independently of one another 0 or 1;
L represents —C(═O)— or —C(—O)—(CH₂)ₖ—C(═O)—, where k is 1, 2 or 3, more particularly 2.

Other compounds of general formula (I) may be coupled with given molecules so as to form prodrugs.

The prodrugs make it possible more particularly to improve the bioavailability of compounds, and particularly to enhance their selectivity for an intended target by enhancing their capacity to pass through cell membranes.

“Coupling” is understood to be the creation of a chemical bond between compounds of formula (I) and other molecules. These bonds may be covalent bonds, ionic or iono-covalent bonds, metallic bonds, hydrogen bonds or van der Waals forces.

According to one particular embodiment, some compounds of general formula (I) may be coupled with hydrophilic molecules.

According to one particular embodiment, some compounds of general formula (I) may be coupled with amphiphilic molecules.

According to one particular embodiment, some compounds of general formula (I) may be coupled with lipophilic molecules.

More particularly, some compounds of general formula (I) may be coupled with fatty acids. Fatty acids suitable for such coupling are saturated fatty acids and unsaturated fatty acids.

Mention may be made particularly of the following saturated fatty acids: caprylic acid (8:0), capric acid (10:0), lauric acid (12:0), myristic acid (14:0), palmitic acid (16:0), stearic acid (18:0), arachidic acid (20:0), behenic acid (22:0), lignoceric acid (24:0) and cerotic acid (26:0).

Mention may also be made of the following unsaturated fatty acids: myristoleic acid (14:1), palmitoleic acid (16:1), sapienic acid (16:1), oleic acid (18:1), elaidic acid (18:1), trans-vaccenic acid (18:1), linoleic acid (18:2), linolelaidic acid (18:2), α-linolenic acid (18:3), γ-linolenic acid (18:3), dihomo-γ-linolenic acid (20:3), arachidonic acid (20:4), eicosapentaenoic acid (20:5), clupanodonic acid (22:5) and docosahexaenoic acid (22:6).

According to another aspect, the invention relates to a pharmaceutical composition or a medicament comprising at least one compound of general formula (I) as defined above, as active principle, and a pharmaceutically acceptable vehicle, said pharmaceutical composition or said medicament being suitable particularly for administration by aerial, oral, parenteral, local, intramuscular or subcutaneous routes.

It should be noted that all of the embodiments set out above with regard to the compound of general formula (I) are also applicable here, on their own or in combination.

According to another aspect, the invention relates to a compound of general formula (I) as defined above, for use thereof for the prevention and/or the treatment of disorders induced by toxins having an intracellular mode of action which utilize retrograde transport, or by viruses or bacteria which utilize retrograde and/or syntaxin 5-dependent transport for infecting the cells, particularly the viruses or bacteria which enter the cells by endocytosis, or by intracellular parasites.

It should be noted that all of the embodiments set out above with regard to the compound of general formula (I) are also applicable here, on their own or in combination.

According to one embodiment, the invention relates to a compound of general formula (I) as defined above, for use thereof for the prevention and/or the treatment of disorders induced by toxins having an intracellular mode of action which utilize retrograde transport. According to one particular embodiment, said toxins having an intracellular mode of action which utilize retrograde transport are chosen from ricin, abrin, Shiga toxin and Shiga-like toxins (Stxs) produced by Shigella dysenteriae (Stx) and E. coli (Stx1 and Stx2, Stx, Stxla, Stx2a, Stx2c, Stx2d, Stx2e as described for example by Melton-Celsa, Microbiol Spectr. 2014; 2(2)), the pertussis toxin (Bordetella pertussis, the agent of whooping cough), subtilase cytotoxin and heat-labile enterotoxin (E. coli).

According to one embodiment, the invention relates to a compound of general formula (I) as defined above, for use thereof for the prevention and/or the treatment of disorders induced by viruses or bacteria which utilize retrograde and/or syntaxin 5-dependent transport for infecting the cells, particularly the viruses or bacteria which enter the cells by endocytosis, or by intracellular parasites, such as the parasites of leishmaniasis.

According to one particular embodiment, the viruses are poxviruses, particularly smallpox virus, monkeypox virus, vaccinia virus and leporipoxviruses, more particularly myxomatosis virus, adeno-associated viruses, more particularly of serotype 2, polyomaviruses, particularly polyomavirus JC and polyomavirus BK, papillomaviruses, filoviruses, particularly Ebola viruses and Marburg virus, enteroviruses, particularly enterovirus 71, herpesviruses, particularly herpes simplex virus type 2 and cytomegalovirus (hCMV), viruses of the genus Arenavirus, particularly lymphocytic choriomeningitis virus, and pneumoviruses, particularly respiratory syncytial virus. According to one particular embodiment, the bacteria are bacteria of the order Chlamydiales, more particularly of the genus Chlamydia, such as Chlamydia trachomatis, Chlamydophila pneumoniae, Chlamydophila psyttaci, or Symkania, such as Symkania negevensis.

According to one particular embodiment, the disorders induced by the intracellular parasites are leishmaniasis, particularly induced by trypanosomes of the genus Leishmania, more particularly Leishmania infantum, Leishmania donovani or Leishmania infantum/donovani hybrid.

The present invention also relates to a method for preventing and/or treating disorders induced by toxins with intracellular activity which utilize retrograde transport, comprising the administration to an individual in need thereof of an effective amount of at least one compound of general formula (I) as defined above.

Lastly, the present invention relates to the use of at least one compound of general formula (I) as defined above for manufacturing a medicament for the treatment of disorders induced by toxins with intracellular activity which utilize retrograde transport.

It should be noted that all of the embodiments set out above with regard to the compound of general formula (I) are also applicable here, on their own or in combination.

According to one particular embodiment, a compound of general formula (I) as defined above may be useful in the treatment of human diseases such as acute intoxications with the toxins ricin or abrin; in the prevention of complications linked to hemolytic uremic syndrome; in the treatment of infections by poxviruses, particularly smallpox, infections with monkeypox, with cowpox, with camelpox or with vaccinia; in the treatment of infections with enteroviruses.

According to one particular embodiment, a compound of general formula (I) as defined above may be useful in the treatment of animal diseases, such as the treatment of myxomatosis in rabbits, of monkeypox in monkeys or of camelpox in camels.

### Synthesis

The compounds of the present invention may be prepared according to methods well known to the person skilled in the art, which include but are not limited to those described below, or by modifications of these methods involving application of standard techniques known to the person skilled in the art of organic synthesis. The modifications and the substitutions which are appropriate will be well known, readily apparent, or readily accessible from the scientific literature to the person skilled in the art. Such methods may be found more particularly in R. C. Larock, Comprehensive Organic Transformations, Wiley-VCH Publishers, 1999.

All of the processes disclosed in the context of the present invention are envisaged to be performed at whatever scale, including the milligram, the gram, the multigram, the kilogram, the multikilogram or the commercial industrial scale.

It should be noted that the compounds of the present invention may comprise an asymmetrically substituted carbon atom, and may be isolated in optically active or racemic forms. Accordingly, all of the chiral, diastereoisomeric, racemic and isomeric forms of a structure are envisaged, unless the stereochemistry or the specific isomeric form is specifically indicated. The preparation and isolation of such optically active forms are well known. For example, the mixtures of stereoisomers may be separated by standard techniques, including but not limited to the resolution of racemic forms, conventional, reversed-phase and chiral chromatography, preferential salt formation, recrystallization, and others, or by chiral synthesis from chiral starting materials, or by deliberate synthesis of the target chiral centers.

The compounds of the present invention may be prepared by diverse synthesis pathways. In the reactions described hereinafter, it may be necessary to protect the reactive functional groups, examples being the hydroxyl, amino or thio groups, when they are present in the end product, in order to prevent them from participating in unwanted secondary reactions. The conventional protective groups may be used in accordance with the common practice: for example, see T. W. Greene and P. G. M. Wuts in Protective Groups in Organic Chemistry, 3rd ed., John Wiley and Sons, 1999; J. F. W. McOmie in Protective Groups in Organic Chemistry, Plenum Press, 1973.

The reactants and the starting compounds are available on the market or easily synthesized by techniques well known to the person skilled in the art. Unless otherwise indicated, all of the substituents are as defined earlier.

The compounds of general formula (I) may be obtained by the following reaction:

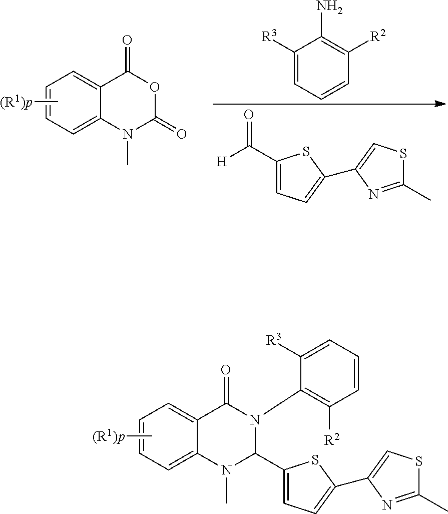

This reaction may particularly be carried out in an organic solvent.

According to one embodiment, the three starting compounds are mixed in an organic solvent, particularly acetic acid.

According to one embodiment, the organic solvent is heated, particularly at reflux or with microwave irradiation, at a temperature, for example, of from 100° C. to 180° C., more particularly from 110° C. to 140° C., more particularly still from 120° C. to 130° C.

The duration of the heating is from 10 minutes to 6 hours, particularly from 30 minutes to 5 hours, more particularly from 1 hour to 4 hours, more particularly still from 2 to 3 hours. R¹ and p are as defined earlier on above.

According to one embodiment, R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, or a halogen atom.

According to one particular embodiment, R² and/or R³ represent groups R²′ and R³′ which correspond to ad hoc protective groups. When R²′ and/or R³′ represent-NO₂, R²′ and/or R³′ are then converted into R² and/or R³═—NH₂, particularly by one of the techniques well known to a person skilled in the art—for example, by the action of zinc in contact with acetic acid.

### Vehicle Solution

The present text also describes a pharmaceutically acceptable vehicle solution.

This vehicle solution additionally enables the solubility of the compounds Retro-2.2 and its analogues to be enhanced for administration by the subcutaneous or oral route, so as to promote their passage in the plasma of mammals. More particularly, the present text describes a pharmaceutically acceptable vehicle comprising compounds of formula (I).

The reason is that the inventors have observed that the majority of the vehicle solutions which exist on the market do not enable the solubility problems of the compounds Retro-2.2 and its analogues to be overcome in a satisfactory manner. Moreover, some vehicles do not allow for all of the charge of molecules to be retained, so giving rise to escape and then undesirable precipitation of these compounds during administration. A vehicle solution as described allows these compounds to be solubilized and/or maintained in fine suspension and so to be administered effectively.

It is shown in the examples that effective solubility of the compounds Retro-2.2, and of its analogues, and more particularly of the compounds of formula (I), is obtained when these compounds are present in a pharmaceutically acceptable vehicle comprising a combination of poppy seed oil fatty acid ethyl esters with castor oil.

In certain embodiments, the pharmaceutically acceptable vehicle comprises (i) from 20% to 40% v/v of poppy seed oil fatty acid ethyl esters and (ii) from 60% to 80% v/v of castor oil.

In certain preferred embodiments, this pharmaceutically acceptable vehicle comprises about 30% of poppy seed oil fatty acid ethyl esters and about 70% of castor oil.

It is also shown in the examples that effective solubility of the compounds Retro-2.2, and of its analogues, and more particularly of the compounds of formula (I), is obtained when these compounds are present in a pharmaceutically acceptable vehicle comprising a combination of dimethyl sulfoxide (DMSO), poppy seed oil fatty acid ethyl esters with castor oil.

Therefore, according to one embodiment, the pharmaceutically acceptable vehicle comprises a combination of dimethyl sulfoxide (DMSO), poppy seed oil fatty acid ethyl esters with castor oil.

In certain embodiments, the pharmaceutically acceptable vehicle comprises (i) from 1% to 5% v/v of dimethyl sulfoxide, (ii) from 20% to 40% v/v of poppy seed oil fatty acid ethyl esters and (iii) from 60% to 75% v/v of castor oil.

In certain particular embodiments, this pharmaceutically acceptable vehicle comprises about 3% of dimethyl sulfoxide, about 29% of poppy seed oil fatty acid ethyl esters, and about 68% of castor oil.

According to one particular embodiment, the poppy seed oil fatty acid ethyl esters are iodized. An example of iodized fatty acid ethyl esters of poppy seed oil is Lipiodol®, which is sold by Guerbet. In this embodiment, the iodized fatty acid ethyl esters are primarily in the form of ethyl monoiodostearate and ethyl diiodostearate.

In one particular embodiment, the poppy seed oil fatty acid ethyl esters are a mixture of poppy seed oil fatty acid ethyl esters in which the fatty acids are chosen from palmitic acid, stearic acid, oleic acid, linoleic acid, linolenic acid, and mixtures thereof. A mixture of poppy seed oil fatty acid ethyl esters which is suitable in a vehicle solution as described may therefore comprise an ethyl ester of palmitic acid, an ethyl ester of stearic acid, an ethyl ester of oleic acid, an ethyl ester of linoleic acid, an ethyl ester of linolenic acid or a mixture of these esters.

In one particular embodiment, the mixture of poppy seed oil fatty acid ethyl esters comprises from 5% to 15% of palmitic acid ethyl esters, from 1% to 5% of stearic acid ethyl esters, from 8% to 15% of oleic acid ethyl esters, from 65% to 75% of linoleic acid ethyl esters, and from 3% to 7% of linolenic acid ethyl esters.

In one preferred embodiment, the mixture of poppy seed oil fatty acid ethyl esters comprises about 10% of palmitic acid ethyl esters, about 2% of stearic acid ethyl esters, about 11% of oleic acid ethyl esters, about 72% of linoleic acid ethyl esters, and about 5% of linolenic acid ethyl esters.

The inventors have discovered surprisingly that the administration of compounds Retro-2.2 and its analogues in a vehicle solution of this type, particularly by the oral or subcutaneous route, enables improvement in the passage of the compounds in the circulation and their concentration in the plasma, and hence their bioavailability in individuals, relative to the vehicles already known in the art.

Moreover, the inventors have discovered that the administration of compounds of formula (I) was also optimized when these compounds were included in a vehicle solution as described above.

It is also shown in the examples that effective solubility of the compounds Retro-2.2, and of its analogues, and more particularly of the compounds of formula (I), is obtained when these compounds are present in a pharmaceutically acceptable vehicle comprising a combination of dimethyl sulfoxide (DMSO) with glycerol triacetate and castor oil.

Therefore, according to one embodiment, the pharmaceutically acceptable vehicle comprises a combination of dimethyl sulfoxide (DMSO), poppy seed oil fatty acid ethyl esters with castor oil.

In certain embodiments, the pharmaceutically acceptable vehicle comprises (i) from 1% to 5% of DMSO, (ii) from 5% to 15% of glycerol triacetate and (iii) from 80% to 94% v/v of castor oil.

In certain particular embodiments, this pharmaceutically acceptable vehicle comprises about 3% of DMSO, about 11% of glycerol triacetate, and about 86% of castor oil.

Vehicle solutions as described are suitable for administration by the aerial, oral, parenteral, local, intramuscular or subcutaneous routes. More particularly, the vehicle solutions are suitable for intramuscular or subcutaneous administration.

As indicated above, the present invention also relates to a pharmaceutical composition or medicament comprising at least one compound of general formula (I) as defined above as active principle, and a pharmaceutically acceptable vehicle, said pharmaceutical composition or said medicament particularly being suitable for administration by the aerial, oral, parenteral, local, intramuscular or subcutaneous routes.

According to one particular embodiment, the pharmaceutically acceptable vehicle is as described above.

Therefore, according to one particular embodiment, the pharmaceutical composition or the medicament comprises, as pharmaceutically acceptable vehicle, (i) from 20% to 40% v/v of poppy seed oil fatty acid ethyl esters and (ii) from 60% to 80% v/v of castor oil.

According to another embodiment, the pharmaceutical composition or the medicament comprises, as pharmaceutically acceptable vehicle, (i) from 1% to 5% v/v of dimethyl sulfoxide, (ii) from 20% to 40% v/v of poppy seed oil fatty acid ethyl esters and (iii) from 60% to 75% v/v of castor oil.

According to another embodiment, the pharmaceutical composition or the medicament comprises, as pharmaceutically acceptable vehicle, (i) from 1% to 5% of DMSO, (ii) from 5% to 15% of glycerol triacetate and (iii) from 80% to 94% v/v of castor oil.

According to one particular embodiment, the composition or the medicament comprises at least one compound of formula (I), more particularly the compound 1, and a pharmaceutically acceptable vehicle comprising (i) from 1% to 5% of DMSO, (ii) from 5% to 15% of glycerol triacetate and (iii) from 80% to 94% v/v of castor oil.

More particularly, the composition or the medicament may comprise at least the(S) enantiomer of the compound 1 ((S)-compound 1) and a pharmaceutically acceptable vehicle comprising (i) from 1% to 5% of DMSO, (ii) from 5% to 15% of glycerol triacetate and (iii) from 80% to 94% v/v of castor oil.

More particularly, the composition or the medicament may comprise at least the(S) enantiomer of the compound 1 ((S)-compound 1) and a pharmaceutically acceptable vehicle comprising (i) about 3% of DMSO, (ii) about 11% of glycerol triacetate and (iii) about 86% v/v of castor oil.

According to one particular embodiment, the pharmaceutical composition or the medicament may take the form of a hydrogel.

More particularly, the pharmaceutical composition or the medicament in the form of a hydrogel may comprise at least one compound of formula (I) and an aqueous solution of PLGA-PEG-PLGA.

The polymer PLGA-PEG-PLGA has been described as forming fluid solutions when it is dissolved in water at low temperature. The PLGA-PEG-PLGA solutions are capable of dissolving hydrophobic compounds. However, on heating to 37° C., the micelles formed by the PLGA-PEG-PLGA chains interact with one another, leading to the formation of a hydrogel. This phenomenon may be exploited in order to generate a fluid formulation of a hydrophobic candidate medicament at ambient temperature, which will spontaneously form a hydrogel on reheating to body temperature (i.e., on subcutaneous injection).

A hydrogel formulation comprising a PLGA-PEG-PLGA polymer of this type has been described in Vinck et al. (In Vivo Sustained Release of the Retrograde Transport Inhibitor Retro-2.1 Formulated in a Thermosensitive Hydrogel. Int. J. Mol. Sci. 2022, 23, 14611. https://doi.org/10.3390/ijms232314611) as enabling administration of the compound Retro-2.1 to mice. This heat-sensitive polymer enabled, particularly, extended release of the compound and better control of its metabolism.

The pharmaceutical composition or the medicament as described are suitable for administration by the aerial, oral, parenteral, local, intramuscular or subcutaneous routes. More particularly, the pharmaceutical composition or the medicament are suitable for intramuscular or subcutaneous administration.

### Definitions

As used in the present description, the term “about” refers to a range of values of +10% of a specific value. By way of example, the expression “about 120 mg” comprises the values of 120 mg±10%, i.e. the values from 108 mg to 132 mg.

In the sense of the present description, the percentages refer to percentages by weight relative to the total weight of the formulation, unless otherwise indicated.

As understood here, the ranges of value in the form of “x-y” or “from x to y” include the endpoints x and y and also the integers lying between these endpoints. By way of example, “1-5” or “from 1 to 5” denotes the integers 1, 2, 3, 4 and 5. The preferred embodiments include each integer taken individually in the value range, and also any sub-combination of these integers. By way of example, the values preferred for “1-5” may comprise the integers 1, 2, 3, 4, 5, 1-2, 1-3, 1-4, 1-5, 2-3, 2-4, 2-5, etc.

As used here, the term “pharmaceutically acceptable salt” refers to salts which, within the scope of a sensible medical judgement, are appropriate for contact with the tissues of human beings and animals, without excessive toxicity, irritation, allergic response or other problematic complications, in proportion with a reasonable benefit/risk ratio.

A pharmaceutically acceptable salt of the compounds of general formula (I) means, particularly, hydrochlorides, hydrobromides, sulfates or bisulfates, phosphates or hydrogenphosphates, acetates, oxalates, benzoates, succinates, fumarates, maleates, lactates, citrates, tartrates, gluconates, methanesulfonates, benzenesulfonates and para-toluenesulfonates.

A halogen atom means the chemical elements of group VII of the Periodic Table of the Elements, particularly fluorine, chlorine, bromine and iodine.

The term “alkyl radical of 1 to 3 or 4 carbon atoms” denotes a linear or branched hydrocarbon radical; examples include methyl, ethyl, propyl, isopropyl or tert-butyl.

“PEG” or “poly(ethylene glycol)” or “polyethylene glycol” means, particularly, groups-(CH2-CH2-O)m-, optionally terminated by a group chosen from H, a C1 to C3 alkyl, or groups —(—O—CH2-CH2)m-, optionally terminated by a group chosen from H, —OH and a C1 to C3 O-alkyl, m being chosen from 1 to 500, more particularly from 4 to 500, particularly from 30 to 60.

The average molar mass of the PEG may particularly be indicated after the term “PEG” after the name: for example, PEG-1450 (1 450 g·mol⁻¹).

“PLA” or “poly(lactic acid)” or “polylactic acid” means, particularly, groups —(C(═O)—CH(CH3)—O)p-, optionally terminated by a group chosen from H, a C1 to C3 alkyl, or groups —(O—CH(CH3)-C(═O))p-, optionally terminated by a group chosen from —OH, and a C1 to C3 O-alkyl, p being chosen from 1 to 2000, particularly from 5 to 500, more particularly still from 10 to 50.

The average molar mass of the PLA may particularly be indicated after the term “PLA” after the name: for example, PLA-1036 (1 036 g·mol⁻¹).

“PLGA” or “PLG” means a poly(lactic acid-co-glycolic acid), and particularly groups —((C(═O)—CH(CH3)-O)p-(C(═O)—CH2-O)q)r-, optionally terminated by a group chosen from H, a C1 to C3 alkyl, or groups —((O—CH2-C(═O))q-(O—CH(CH₃)—C(═O))p)r-, optionally terminated by a group chosen from —OH, and a C1 to C3 O-alkyl, p, q and r being chosen such that the PLGA polymer is a random or block, more particularly random, polymer, and particularly such that the average molar mass of the PLGA, for example indicated after the term “PLGA” after the name, is from 200 to 4000, particularly from 800 to 1200 g·mol⁻¹. p, q and r are particularly such that the molar percentage of lactic acid in the PLGA is about 20%. More particularly, p, q and r are such that the PLGA consists of 12 units of lactic acid and 3 units of glycolic acid.

### Example

### I. Synthesis of Compounds According to the Invention

All of the chemical products and solvents used in the syntheses are of reagent grade and were used without further purification. The CH₂Cl₂ was distilled over calcium hydride before use. The glassware was flame-dried under vacuum and cooled under nitrogen to ambient temperature. All of the reactions were performed under dry nitrogen and checked by TLC.

Purification was carried out particularly on a CombiFlash with a UV-vis detector and RediSep columns. The samples particularly were adsorbed on Celite or silica and loaded into solid-charge cartridges. An ethyl acetate/cyclohexane or methanol/dichloromethane gradient was used, particularly. The fractions were collected, particularly, on the basis of detection at 254 nm.

Analysis and purification by HPLC-MS were performed, particularly, by means of a Waters system (binary gradient module 2525, in-line degassing unit, sample manager 2767, photodiode array detector 2996) with a binary system for the solvent gradient. The eluent was more particularly a gradient of (99.9% water/0.1% HCOOH) and (99.9% MeCN/0.1% HCOOH) or (99.9% water/0.1% HCOOH) and (99.9% MeOH/0.1% HCOOH). Each compound was applied to a Zorbax SB-C18 column of 100-4.6 mm (5 mm) equilibrated with H₂O/MeCN or H₂O/MeOH 95:5. This system was coupled for example to a Waters Micromass ZQ system with a ZQ2000 quadrupole analyzer. Ionization was carried out by electrospray and the other parameters were as follows: source temperature 120° C., cone voltage 20 V, and continuous injection of the sample at a flow rate of 0.3 mL per min. The mass spectra were recorded in positive and negative ion mode in the 100-2000 m/z range and processed with the Mass Lynx 4.0 software.

The infrared spectra were recorded particularly on a Spectrum Two equipped with an UATR Two (Perkin Elmer), diamond/ZnSe (1 reflection).

The NMR analyses were carried out particularly on a Bruker Avance 400 Ultrashield spectrometer. The 1H-NMR and 13C spectra were recorded at ambient temperature and at 400 MHz and 100 MHz respectively; the samples were dissolved in DMSO-d6 or CDCl₃ at a concentration of about 5 mM. The DMSO singlet signal was regulated at 2.50 ppm. The chemical shifts are given in ppm and the coupling constants in Hz. The spectral data are consistent with the associated structures.

### Preparation of the compound 1: Synthesis of 3-(2,6-dihydroxyphenyl)-6-fluoro-1-methyl-2-(5-(2-methylthiazol-4-yl)thiophen-2-yl)-2,3-dihydroquinazolin-4(1H)-one

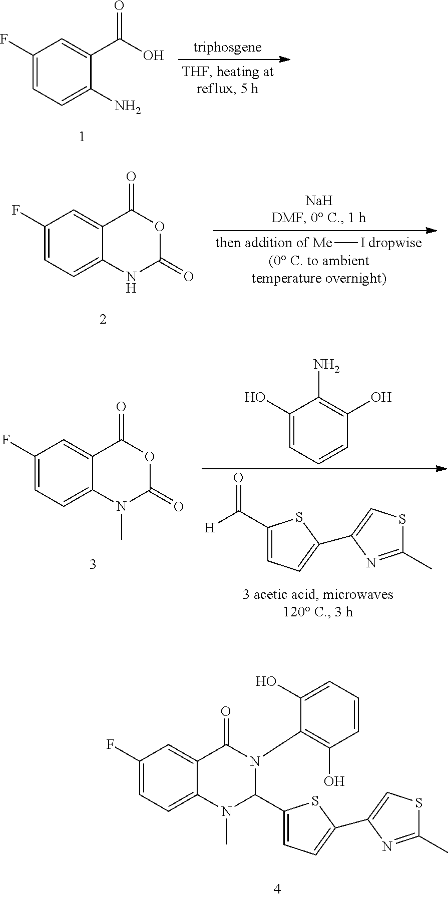

TABLE 1

| | | M | n | m | V |
|---|---|---|---|---|---|
| Reactants | eq. | (g/mol) | (mmol) | (mg) | (mL) |
| 5-Fluoro-N- | 1 | 195.03 | 1 | 195 | / |
| methylisatoic anhydride | | | | | |
| 2-Amino-1,3-benzenediol | 1.5 | 125 | 1.50 | 187 | / |
| 5-(2-Methyl-1,3-thiazol-4-yl)- | 1 | 209.00 | 1 | 209 | / |
| 2-thiophenecarbaldehyde | | | | | |
| Acetic acid | / | / | / | / | 2 |

N-Methyl-5-fluoroisatoic anhydride (195 g/mol, 0.195 g, 1 mmol, 1 equiv.) obtained as described in Gupta et al. (ACS Med. Chem. Lett. 2014, 5, 1, 94-97), 2-amino-1,3-benzenediol (125 g/mol, 0.187 g, 1.5 mmol, 1.5 equiv.) and commercially available 5-(2-methyl-1,3-thiazol-4-yl)-2-thiophenecarbaldehyde (209 g/mol, 0.209 g, 1 mmol, 1 equiv.) were dissolved in acetic acid (2 mL). The mixture was heated in a microwave synthesizer at 120° C. for 3 h. The crude mixture was diluted in ethyl acetate. The organic phase was washed with saturated aqueous NaHCO₃ solution and distilled water and then dried over magnesium sulfate and filtered, and the solvent was evaporated under reduced pressure. Purification on a chromatography column on silica gel, using a cyclohexane/ethyl acetate (90/10 to 70/30) elution mixture, enabled recovery of the compound 4 from the scheme above in the form of a yellowish powder (467.5 g/mol, 0.467 g, quantitative). This compound corresponds to the compound 1 according to the invention.

1H NMR (DMSO-d6): 2.61 (s, 3H); 2.83 (s, 3H); 5.96 (s, 1H); 6.21 (dd, 1H, J=1.1, J=8.2); 6.42 (dd, 1H, J=1.1, J=8.1); 6.79 (dd, 1H, J=4.2, J=9), 6.86 (d, 1H, J=3.7); 6.9 (t, 1H, J=8.2); 7.21 (d, 1H, J=3.6); 7.33 (td, 1H, J=3.2, J=8.8); 7.51 (dd, 1H, J=3.1, J=8.8); 7.66 (s, 1H); 9.03 (s, 1H); 9.82 (bs, 1H).

UPLC/MS: Retention time: 2.95 min,

M+H=468.9

### Preparation of the compound 2: Synthesis of 3-(2,6-diaminophenyl)-6-fluoro-1-methyl-2-(5-(2-methylthiazol-4-yl)thiophen-2-yl)-2,3-dihydroquinazolin-4(1H)-one

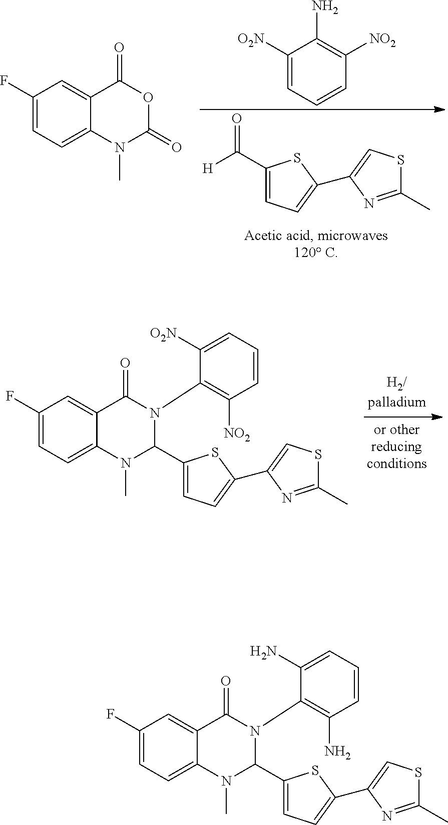

The compound 2 according to the invention may be obtained similarly by following a synthesis protocol as described above for the compound 1, following the synthesis scheme above.

II. Measurement for Evaluating the Protective Activity of the Compounds of the Invention with Regard to Various Toxins and Viruses

### 1. With Regard to Shiga Toxin, Ricin and Abrin

### Protocol and Calculation of the EC₅₀

The compounds were tested either on A549 cells (human pulmonary epithelial cells) or on HeLa cells (human uterine cancer cells), against the Shiga toxins (Stx-1 and/or Stx-2), against ricin, or against abrin. The human cells are cultured at 37° C. in an atmosphere containing 5% CO₂ in 150 cm² culture flasks in the medium DMEM (Dulbecco's Modified Eagle Medium) containing 100 U/mL of penicillin and 100 μg/mL of streptomycin. The cells are seeded at a density of 50 000 cells per well into Cytostar-T 96-well plates with a solid scintillant in the base. The cells (100 μL in complete DMEM: DMEM+10% fetal calf serum, FCS) are preincubated or not with the inhibitors (50 μL; various concentrations, preincubation for 3 h). The inventive compound 1 was prepared in accordance with the present protocol above. The non-inventive compounds A to J were prepared in accordance with the protocols presented in international patent applications WO2014/060586 A1 or WO2020/109510 A1.

The structure of these compounds is indicated in the table below:

TABLE 2 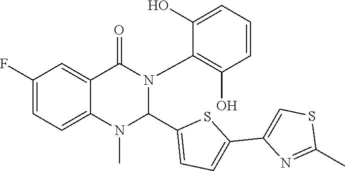 [SMILES: Cc1nc(-c2ccc(C3N(C)c4ccc(F)cc4C(=O)N3c3c(O)cccc3O)s2)cs1]
Compound 1: (inventive) 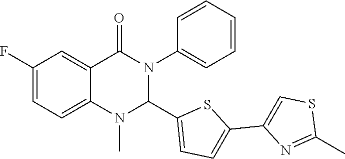 [SMILES: Cc1nc(-c2ccc(C3N(C)c4ccc(F)cc4C(=O)N3c3ccccc3)s2)cs1]
Compound A (non-inventive) 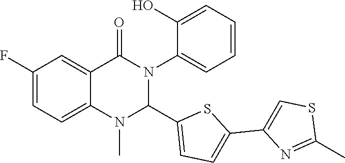 [SMILES: Cc1nc(-c2ccc(C3N(C)c4ccc(F)cc4C(=O)N3c3ccccc3O)s2)cs1]
Compound B (non-inventive) 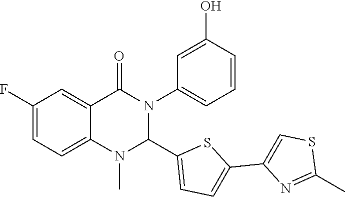 [SMILES: Cc1nc(-c2ccc(C3N(C)c4ccc(F)cc4C(=O)N3c3cccc(O)c3)s2)cs1]
Compound C (non-inventive) 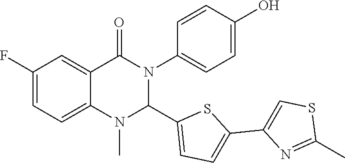 [SMILES: Cc1nc(-c2ccc(C3N(C)c4ccc(F)cc4C(=O)N3c3ccc(O)cc3)s2)cs1]
Compound D (non-inventive) 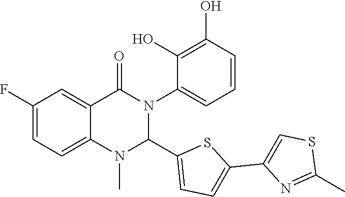 [SMILES: Cc1nc(-c2ccc(C3N(C)c4ccc(F)cc4C(=O)N3c3cccc(O)c3O)s2)cs1]
Compound E (non-inventive) 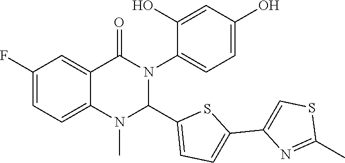 [SMILES: Cc1nc(-c2ccc(C3N(C)c4ccc(F)cc4C(=O)N3c3ccc(O)cc3O)s2)cs1]
Compound F (non-inventive) 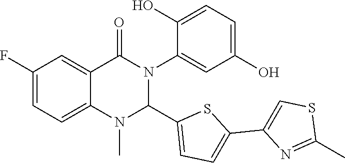 [SMILES: Cc1nc(-c2ccc(C3N(C)c4ccc(F)cc4C(=O)N3c3cc(O)ccc3O)s2)cs1]
Compound G (non-inventive) 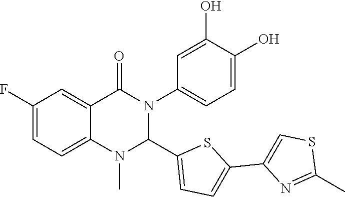 [SMILES: Cc1nc(-c2ccc(C3N(C)c4ccc(F)cc4C(=O)N3c3ccc(O)c(O)c3)s2)cs1]
Compound H (non-inventive) 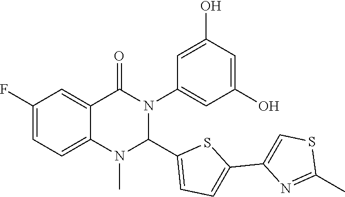 [SMILES: Cc1nc(-c2ccc(C3N(C)c4ccc(F)cc4C(=O)N3c3cc(O)cc(O)c3)s2)cs1]
Compound I (non-inventive) 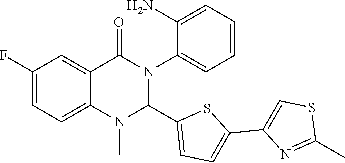 [SMILES: Cc1nc(-c2ccc(C3N(C)c4ccc(F)cc4C(=O)N3c3ccccc3N)s2)cs1]
Compound J (non-inventive)

These compounds, in powder form, were subsequently dissolved in pure DMSO at a concentration of 10 mM. This solution is subsequently diluted in the culture medium and used in the examples hereinafter in a range of concentrations. The complete medium, made up with Shiga toxin, ricin or abrin (50 μL, variable concentration range), is subsequently added to each well. After incubation for 20 h, the medium (200 μL) is removed and replaced by a DMEM medium without leucine (Eurobio) containing 10% of FCS and 0.5 μCi/mL of [¹⁴C]-leucine (GE). After incubation for 7 h at 37° C., the incorporation of radioactivity by the cells is determined by reading the plates using a Wallac 1450 Microbeta trilux scintillation counter (PE).

Since these toxins block the synthesis of proteins, the cells affected are no longer capable of incorporating the radiolabeled leucine into their proteins. Conversely, the cells treated with inhibitors still synthesize proteins and therefore do incorporate the radiolabelled amino acid. As the cells concentrate the radioelement sufficiently close to the base of the well, this causes excitation of the scintillant contained in the plates and leads to the emission of photons, which is detected by the scintillation counter (measurement in counts per minute, cpm). These data are subsequently expressed as a percentage of protein synthesis by the cells. The cytotoxicity curves can therefore be drawn, without inhibitor or in the presence of an inhibitor. The analysis of the data by nonlinear regression allows estimation of the IC₅₀ in the absence or presence of compound, being the concentration of toxin for which 50% assimilation of radioactive leucine is observed, corresponding to 50% of viable cells. The greater the value of the IC₅₀, the greater the cell protection, as a higher concentration of toxin is then needed in order to generate the same cytotoxicity.

The curve of the IC₅₀ values as a function of the concentrations of compound allows the EC₅₀ to be calculated, representing the concentration of product giving 50% of its maximum antitoxin protective effect. The lower the EC₅₀, the greater the efficacy of the compound.

### 2. With Regard to the Poxviruses Cowpox and Myxomatosis

### Cytotoxicity of the Compounds

The cells RK13 (ATCC CCL-37) (for the myxomatosis virus) and HeLa (ATCC-CCL2) (for the cowpox virus) are cultivated in DMEM without phenol red (D1145; Sigma Aldrich) admixed with 10% FCS (Eurobio-Scientific), 1 mM sodium pyruvate (S8636; Sigma Aldrich), L-glutamine (G7513; Sigma Aldrich) and a penicillin-streptomycin solution (P0781; Sigma Aldrich).

The cells are seeded at between 6000 and 8000 cells per well in a Corning Cellbind 96-well plate in complete DMEM medium. 24 h after seeding, the cells are treated with the compounds 1 and A to J for a series of concentrations. The cells are subsequently incubated at 37° C. and 5% CO₂ for 3 to 6 days.

A time after treatment, the cells are labeled with Image-iT DEAD Green Viability Stain (Invitrogen I10291) and with MitoTracker Orange (Invitrogen M7510) for 30 minutes at 37° C. The cells are fixed with 4% formalin (Sigma) for 10 min, washed with PBS and incubated with Hoechst 33342 PBS (1 mg/mL).

The acquisition of the data by high-content microscopy is carried out on a Thermo CellInsight CX7 HCS microscope, utilizing a compartmental analysis algorithm. The results are extracted, normalized relative to the non-treated conditions, and expressed as the mean of three independent wells+/−SD.

The 50% cytotoxic concentration (CC₅₀), which corresponds to the concentration of compound administered that reduces the viability of the cells by 50%, was determined for each compound. The lower the value of the CC₅₀, the more toxic the compound for the cells.

### Anti-Viral Activity

The anti-viral activity is measured using the ANCHOR technology (NeoVirTech). The cells are cultivated under the same conditions as for the cytotoxicity test, treated at 8 concentrations in triplicate and infected with the myxomatosis-derived virus MYXV-T1-ANCHOR (MOI 0.5) and the cowpox-derived virus VacV-ANCHOR (MOI 0.1).

3 to 4 days after infection, the cells are fixed under the same conditions as described above, and incubated with Hoechst 33342 PBS (1 mg/mL).

The plates are imaged with a Thermo Scientific CellInsight CX7 HCS microscope. Compartmental analysis coupled with a Spot Detector algorithm is utilized in order to detect and quantify the degree of infection (number of fluorescent cells to total number of cells) and the degree of replication (intensity of the ANCHOR spots).

The results are obtained after measurement and automated analysis of a minimum of 2000 cells per well per replicate. The results are extracted, normalized relative to the infected/non-treated conditions, and expressed as the mean of three independent wells+/−SD.

In the same way as in section II.1., the EC₅₀ was determined for each compound.

### 3. Results

The EC₅₀ results for each of the compounds with regard to the toxins and the viruses indicated are given in the table below.

TABLE 3

| | EC₅₀ in vitro (nM) | | | | | | CC₅₀ in vitro (nM) | | MW | Log |
|---|---|---|---|---|---|---|---|---|---|---|
| Names | Stx1 | Stx2 | Ricin | Abrin | VACV | MYXV | HeLa | Rk13 | (Da) | S |
| Compound 1 | 16 | 17 | 18 | 11 | 0.78 | 6 | 57 700 | 18 900 | 467.5 | −5.33 |
| (inventive) | (n = 5) | (n = 5) | (n = 3) | (n = 3) | | | | | | |
| Compound A | 52 | 60 | 65 | 50 | 37.8 | 74.5 | >50 000 | 3 350 | 435.5 | −6.29 |
| (non- | (n = 2) | (n = 4) | (n = 3) | (n = 2) | | | | | | |
| inventive) | | | | | | | | | | |
| Compound B | 25 | 22 | 26 | 35 | 3.6 | 19 | 39 900 | 14 000 | 451.5 | −5.81 |
| (non- | (n = 8) | (n = 8) | (n = 3) | (n = 3) | | | | | | |
| inventive) | | | | | | | | | | |
| Compound C | 62 | 71 | 55 | 48 | 32.2 | 85.8 | 29 200 | 3 510 | 451.5 | −5.83 |
| (non- | (n = 2) | (n = 2) | (n = 3) | (n = 3) | | | | | | |
| inventive) | | | | | | | | | | |
| Compound D | 483 | | | | 628 | 5 800 | 20 400 | 12 900 | 451.5 | −5.83 |
| (non- | | | | | | | | | | |
| inventive) | | | | | | | | | | |
| Compound E | 32 | 35 | 47 | 44 | 4.8 | 11.5 | 8 500 | 1 300 | 467.5 | −5.35 |
| (non- | (n = 2) | (n = 2) | (n = 3) | (n = 2) | | | | | | |
| inventive) | | | | | | | | | | |
| Compound F | 1500 | | | | 27.3 | | 3 600 | | 467.5 | −5.34 |
| (non- | | | | | | | | | | |
| inventive) | | | | | | | | | | |
| Compound G | 642 | | | | 19 | 237 | 36 300 | 12 600 | 467.5 | −5.36 |
| (non- | | | | | | | | | | |
| inventive) | | | | | | | | | | |
| Compound H | 2500 | | | | | | | | 467.5 | −5.36 |
| (non- | | | | | | | | | | |
| inventive) | | | | | | | | | | |
| Compound I | >30000 | | | | 7 300 | 9 751 | 25 900 | 20 100 | 467.5 | −5.36 |
| (non- | | | | | | | | | | |
| inventive) | | | | | | | | | | |
| Compound J | 21 | 27 | 18 | 20 | 1 | 1.44 | >50 000 | 7 460 | 450.5 | −6.07 |
| (non- | (n = 3) | (n = 3) | (n = 2) | (n = 2) | | | | | | |
| inventive) | | | | | | | | | | |

These results show that the compounds of formula (I), and more particularly the compound 1 according to the invention, have very low EC₅₀s. They exhibit improved properties with regard to cell protection against Shiga toxins, ricin and abrin and against the cowpox and myxomatosis viruses, particularly relative to equivalent compounds of the prior art.

It is demonstrated, moreover, that the compounds of formula (I), and more particularly the compound 1 according to the invention, have high CC₅₀ values, which are synonymous with a low cytotoxicity of these compounds with regard to the treated cells.

Lastly, the calculated solubility expressed as log S was estimated by calculation in silico for each of the compounds. The higher the value of log S, the greater the solubility of the molecule. These results show that the compounds according to the invention, particularly the compound 1, exhibit an improved solubility.

In conclusion, these results show that compounds according to the invention, such as the compound 1, exhibit both an effective inhibitory activity with regard to the various Shiga toxins Shiga Stx-1, Stx-2, ricin and abrin, and with regard to the various pox viruses of cowpox and myxomatosis, without presenting a cytotoxicity problem for the treated cells. Moreover, these features, particularly of solubility, are improved with the compounds according to the invention, relative to those obtained for compounds of the prior art.

### III. Measurement of the Pharmacokinetic Activity of Analogues of Retro-2.2 and of Compounds of Formula (I) in a First Vehicle Solution (DMSO/Poppy Seed Oil Fatty Acid Ethyl Esters/Castor Oil)

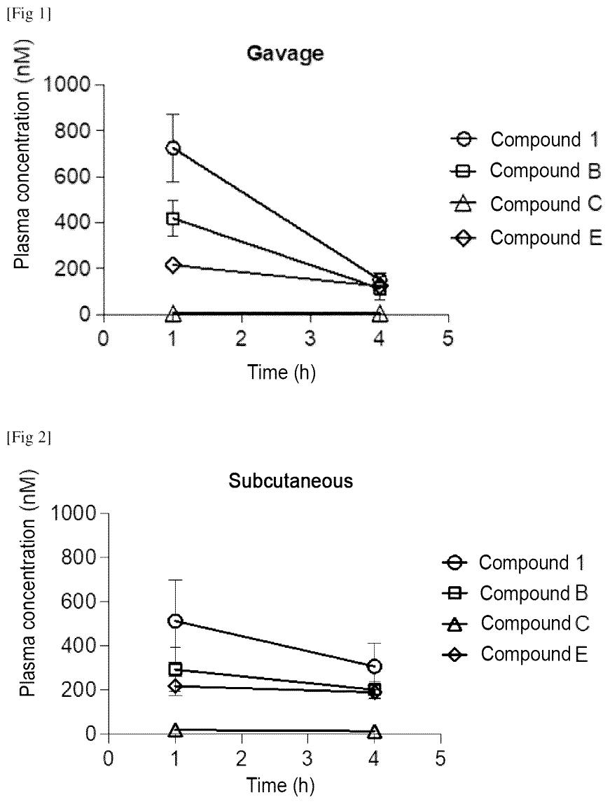

The pharmacokinetic activity (concentration of the molecule in the plasma (nM) as a function of time (h)) was tested for the following analogues of the molecule Retro-2.2: compound 1 (white circles), Retro-2.2 (compound B from the table above) (white squares), compound C (white triangles) from the table above, and compound E (white diamonds), also noted in the table above. The compounds were administered to the female BALB/c mouse by the oral route (gavage) (FIG. 1) and by the subcutaneous route (FIG. 2) at a concentration of 33 mg/kg in a mixture of Lipiodol®/castor oil in proportions by mass of 30%/70%. The plasma concentration of these compounds was measured by liquid chromatography with mass spectrometry in tandem (LC-MS-MS).

The results of these tests show that the compound 1 according to the invention exhibits the best pharmacokinetic properties of all of the molecules tested, since it allows the best exposure in terms of concentration of the molecule, and does so for a prolonged period. Moreover, it is observed that the relative bioavailability of the compound 1 according to the invention is better when it is administered by the subcutaneous route, since the area under the curve is greater compared to oral administration.

Furthermore, the pharmacokinetic activity of the compound Retro-2.2 was compared to that of the compound 1 according to the invention following administration by the subcutaneous route to the female BALB/c mouse at a concentration of 33 mg/kg in a vehicle solution comprising a mixture of Lipiodol®/castor oil in proportions by mass of 30%/70% (formulation B) or in a vehicle solution as described in the text comprising a mixture of DMSO/Lipiodol®/castor oil in proportions by mass of 3%/29%/68% (formulation C).

To prepare the formulations C, the molecules Retro-2.2 and the compound 1 according to the invention, in powder form, were dissolved in pure DMSO. The solution was homogenized by vortexing for 30 seconds at maximum speed. The Lipiodol® was subsequently added. The solution was again homogenized by vortexing for 30 seconds at maximum speed. The castor oil was added gently with a pipette. The solution was homogenized by inverting the tube containing it 20 times. It then underwent sonication in a water bath at 37° C. for 30 minutes. Again, the solution was homogenized by inverting the tube containing it 20 times.

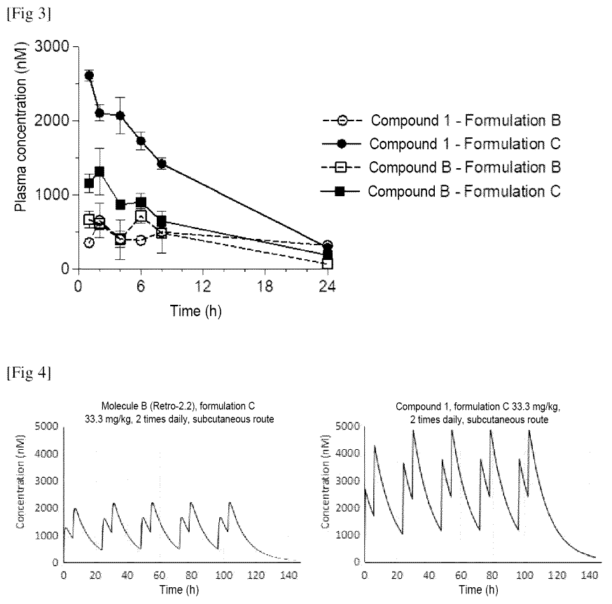

The results are reported in FIG. 3 according to the following representation: Retro-2.2 in formulation B (white squares), Retro-2.2 in formulation C (black squares), compound 1 according to the invention in formulation B (white circles) and compound 1 according to the invention in formulation C (black circles).

The results of these tests show that both for the molecule Retro-2.2 and for the compound 1 according to the invention, the relative bioavailability is improved when the molecule is administered in the formulation C. Moreover, these results show that the compound 1 according to the invention exhibits an even better relative bioavailability than that of the molecule Retro-2.2, irrespective of the vehicle solution used.

Lastly, a simulation of repeated administration of the molecule Retro-2.2 and of the compound 1 according to the invention was produced on the basis of the results obtained from the pharmacokinetic parameters of the tests of a single administration. These tests were carried out by subcutaneous administration to the female BALB/c mouse, at a concentration of 33 mg/kg, twice daily in a formulation C comprising a mixture of DMSO/Lipiodol®/castor oil in proportions by mass of 3%/29%/68%.

The pharmacokinetic parameters obtained with Retro-2.2 or the compound 1 according to the invention in a formulation C, administered by the subcutaneous route, were used for simulating the plasma concentrations which would be obtained following two subcutaneous injections per day, 6 hours apart (FIG. 4).

The results show that the EC₅₀ of Retro-2.2 against the toxin ricin, abrin or Stx is about 20 or 30 nM. For a compound 1 according to the invention, the EC₅₀ is around or less than 20 nM. Consequently, the simulation suggests that twice-daily subcutaneous injections of Retro-2.2 or of a composition according to the invention should enable the attainment of concentrations largely 10 times greater than their respective ECsos against these toxins for a good part of the day. This criterion must be reached in order to validate a molecule in early studies of development of medicaments.

The same applies with regard to their activity toward pox viruses.

### IV. Measurement of the Pharmacokinetic Activity of Analogues of Retro-2.2 and of Compounds of Formula (I) in a Second Vehicle Solution (DMSO/Glycerol Triacetate/Castor Oil)

The pharmacokinetic activity (concentration of the molecule in the plasma (μM) as a function of time (h)) of the(S) enantiomer of the compound 1 according to the invention was tested. The compound was administered to the female BALB/c mouse by the subcutaneous route at a concentration of 33 mg/kg in a mixture of DMSO 3%/Glycerol triacetate 11%/Castor oil 86%. The plasma concentration of the compound was measured by liquid chromatography with mass spectrometry in tandem (LC-MS-MS).

The pharmacokinetic parameters calculated are provided in the table hereinafter.

TABLE 4
Pharmacokinetic parameters of (S)-compound 1

| | |
|---|---|
| Elimination rate (1/h) | 0.06 |
| T_{1/2} (h) | 12.29 |
| Tₘₐₓ (h) | 6 |
| Cₘₐₓ (μM) | 0.86 |
| Area under the curve (AUC₀₋ₜ) (μM*h) | 17.30 |
| Mean residence time (MRT₀₋ₜ) (h) | 19.13 |
| Distribution volume/F (L/kg) | 67.38 |
| Clearance/F (L/kg/h) | 3.80 |

The results of these tests show that(S)-compound 1 (the(S) enantiomer of the compound 1) exhibits particularly satisfactory pharmacokinetic properties and does so for a prolonged period.

Subsequently, the compound 1 and also other above-cited analogues of the molecule Retro-2.2 (compound A, compound B, compound C and compound E) were incubated with hepatic microsomes of female CD1 mice in the presence or in the absence of NADPH, the cofactor of CYP450s, at a final concentration of 5 μM for 45 minutes at 37° C. and 300 rpm (Thermomixer). Samples were taken at various times and admixed with cold acetonitrile to halt the reaction and precipitate the proteins. The samples were centrifuged and the supernatant was diluted and analyzed by LC-MS-MS in the presence of a known concentration of Retro-2.1 as internal standard.

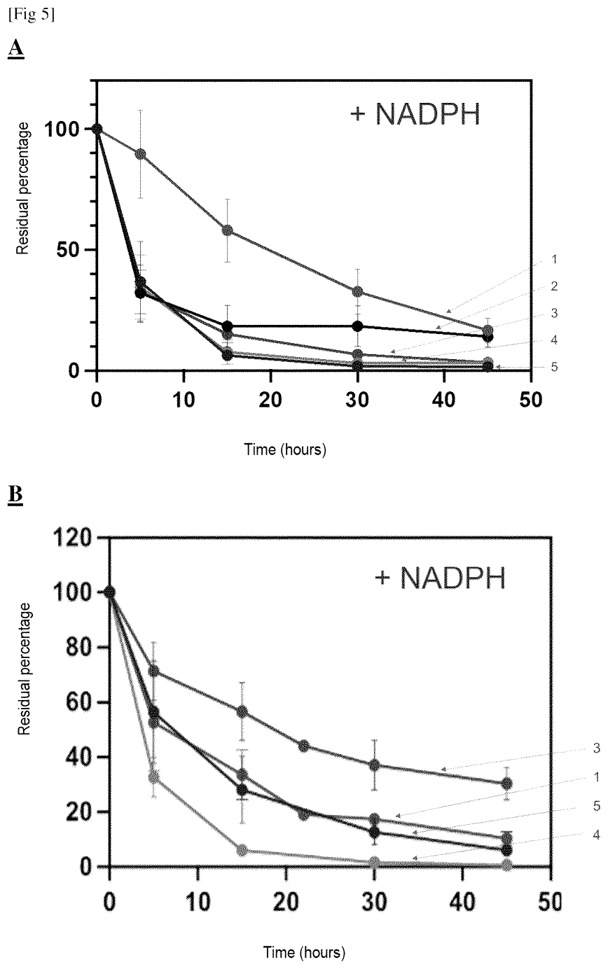

As shown by the results in FIG. 5, the compound 1 exhibits a slowed metabolism relative to the other compounds, more particularly in the presence of hepatic mouse microsomes. The compounds are stable in the absence of NADPH (results not presented).

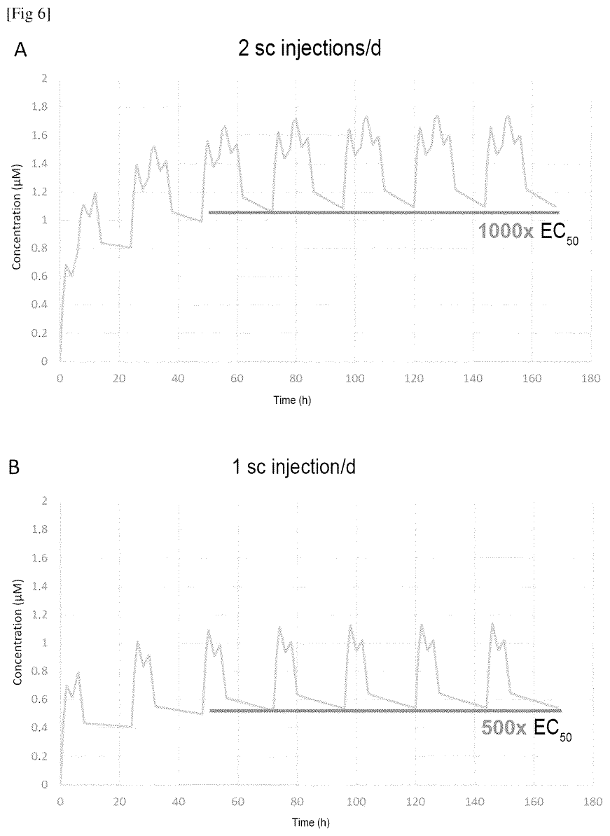

From the experimental curves, the inventors subsequently undertook a simulation of repeated injections of the(S) enantiomer of the compound 1 at a dose of 33 mg/kg once or twice daily. These simulations enabled the attainment of a theoretical plasma concentration of 500 times and 1000 times the EC₅₀ of (S)-compound 1 in vitro (FIG. 6).

Afterward, the subcutaneous injection of (S) enantiomer of the compound 1 at the dose of 33 mg/kg to the Balb/c mouse enabled a concentration of compound in the organs to be obtained of between 67 and 350 times the EC₅₀ against the Shiga toxins of (S)-compound 1 in vitro. At the doses of 1 to 3 mg/kg, once to twice daily, the plasma and pulmonary concentrations were reached of 75 to 175 nM, i.e. between 12 and 29 times the EC₅₀ of (S)-compound 1 in vitro. They were from 50 to 125 nM in the lungs after 2 h, i.e. between 8 and 21 times the EC₅₀ of (S)-compound 1 in vitro.

The compound 1 was identified in vivo in various organs of the mice treated subcutaneously at 33 mg/kg as described above, namely in the brain, in the lungs, in the liver, in the small intestine, in the colon, in the kidneys and in the plasma. Two hours after the administration of the compound 1, the concentrations of compound 1 as measured by LC-MS-MS were as follows:
brain: 1.7 μM
lungs: 0.5 μM
liver: 0.6 μM
small intestine: 2.1 μM
colon: 0.8 μM
kidneys: 0.4 μM
plasma: 0.7 μM

These results show that the administration of compound 1 by the subcutaneous route, more particularly in the formulation DMSO 3%/Glycerol triacetate 11%/Castor oil 86%, enables a concentration to be obtained which is theoretically protective to mice against the pox viruses or the Shiga toxins.

### V. Metabolite of a Compound of Formula (I)

The structure of the majority metabolite of the compound 1 identified in the culture supernatants after incubation in the presence of hepatic microsomes (see above) was determined by LC-MS-MS and then synthesized for confirmation by the methods known in the art to the person skilled in the art—for example, according to an operating mode similar to the synthesis process described in Forrester et al. (Functional dissection of the retrograde Shiga toxin trafficking inhibitor Retro-2. Nat Chem Biol. 2020 March; 16(3):327-336. doi: 10.1038/s41589-020-0474-4. Epub 2020 Feb. 17. PMID: 32080624; PMCID: PMC7039708).

The compound obtained is as follows:

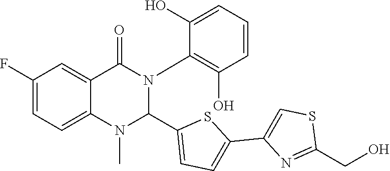 [SMILES: CN1c2ccc(F)cc2C(=O)N(c2c(O)cccc2O)C1c1ccc(-c2csc(CO)n2)s1]

This compound, also called metabolite, was also identified in vivo in the kidney, the liver, the colon, the lungs and the brain of female BALB/c mice after subcutaneous treatment with the compound 1 in a concentration of 33 mg/kg in a DMSO/poppy seed oil fatty acid ethyl esters/castor oil solution as described in example III. Two hours after treatment, the following concentrations of compound 1 were measured by LC-MS-MS in the various organs:
kidney: 0.5 μM
liver: 1 μM
colon: <200 nM
lungs: <200 nM
brain: 1.5 μM

1. A compound of general formula (I): 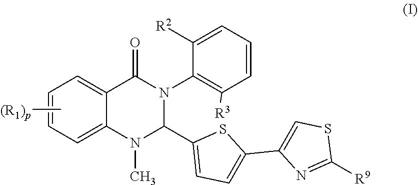
in which
p is 1, 2 or 3;
R¹ represents at each occurrence, independently, a hydrogen atom, a halogen atom, an alkoxy radical of 1 to 3 carbon atoms —NO₂, or —NH₂;
R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, or a halogen atom;
R⁴, R⁵ and R⁶ represent independently of one another a group of formula (II) -L-(X)i-(PEG)-(Y)j-Z, in which:
i and j represent independently of one another 0 or 1;
L represents —C(═O)— or —C(═O)—(CH₂)ₖ—C(═O)—, with k being 1, 2 or 3;
R⁷ and R⁸ represent, independently of one another, a linear or branched C₁ to C₃ alkyl;
X and Y represent independently of one another a poly(lactic acid) or a poly(lactic acid-co-glycolic acid);
PEG represents a poly(ethylene glycol);
Z represents a group chosen from H, a C₁ to C₃ alkyl, —OH, a C₁ to C₃ O-alkyl, or -L-Re, in which L is defined as above and Re is a residue of formula (I) connected to the group -L-(X)i-(PEG)-(Y)j-defined above by way of its group R² or R³, the group R² or R³ being —OH, —NH₂ or —SO₂—NH₂;
R⁹ represents —CH₃ or —CH₂—OH,
and also the stereoisomeric forms, the mixtures of stereoisomeric forms, or the pharmaceutically acceptable salts thereof.

2. The compound of general formula (I) as claimed in claim 1, wherein R⁹ represents —CH₃.

3. The compound of general formula (I) as claimed in claim 1, wherein p is 1.

4. The compound of general formula (I) as claimed in claim 1, wherein R¹ represents a halogen atom.

5. The compound of general formula (I) as claimed in claim 1, wherein R² and R³ represent independently of one another a group chosen from —OH, —OCH₃, —SH, —SCH₃, —OR⁴, —NH₂, —NHR⁵, —NR⁷R⁸, —SO₂—NH₂, —SO₂—NH—R⁶, SO₃H, where R⁴, R⁵, R⁶, R⁷ and R⁸ are as defined in claim 1.

6. The compound of general formula (I) as claimed in claim 1, of general formula (Ia) below: 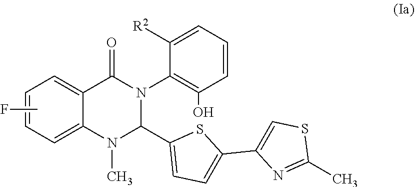
in which R² is as defined in claim 1.

7. The compound of general formula (I) as claimed in claim 1, of general formula (Ib) below: 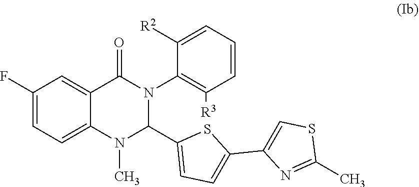
wherein R² and R³ are chosen from the following combinations: R² is an —OH group and R³ is an —NH₂ group, R² is an —OH group and R³ is an —SO₂NH₂ group, R² is an —OH group and R³ is an —SH group, R² is an —OH group and R³ is an —SO₃H group, R² is an —OH group and R³ is an —SMe group, R² is an —OH group and R³ is an —OMe group, R² is an —OH group and R³ is a halogen atom, R² is an —NH₂ group and R³ is an —NH₂ group, R² is an —NH₂ group and R³ is an —SO₂NH₂ group, R² is an —NH₂ group and R³ is an —SH group, R² is an —NH₂ group and R³ is an —SO₃H group, R² is an —NH₂ group and R³ is an —SMe group, R² is an —NH₂ group and R³ is an —OMe group, R² is an —NH₂ group and R³ is a halogen atom, R² is an —SH group and R³ is an —SH group, R² is an —SH group and R³ is an —SO₂NH₂ group, R² is an —SH group and R³ is an —SO₃H group, R² is an —SH group and R³ is an —SMe group, R² is an —SH group and R³ is an —OMe group, R² is an —SH group and R³ is a halogen atom, R² is an —OMe group and R³ is an —OMe group, R² is an —OMe group and R³ is an —SMe group, R² is an —OMe group and R³ is a halogen atom, R² is an —SMe group and R³ is an —SMe group, R² is an —SMe group and R³ is a halogen atom, and R² is a halogen atom and R³ is a halogen atom where Me is a methyl.

8. The compound of general formula (I) as claimed in claim 1, of formula below:

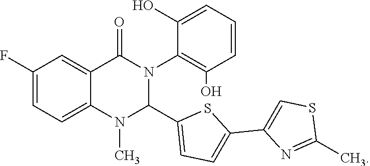

9. A method for preventing and/or treating disorders induced by toxins having an intracellular mode of action which utilize retrograde transport, or by viruses or bacteria which utilize retrograde and/or syntaxin 5-dependent transport for infecting the cells, or by intracellular parasites, the method comprising administering to a patient in need thereof the compound of general formula (I) as claimed in claim 1.

10. The method as claimed in claim 9, wherein the toxins having an intracellular mode of action which utilize retrograde transport are chosen from ricin, abrin, Shiga toxin and the Shiga-like toxins produced by Shigella dysenteriae and E. coli, the pertussis toxin, subtilase cytotoxin and heat-labile enterotoxin.

11. The method as claimed in claim 9, wherein the viruses are poxviruses.

12. A pharmaceutical composition or a medicament comprising at least one compound of general formula (I) as defined in claim 1 as active principle, and a pharmaceutically acceptable vehicle, the pharmaceutical composition or the medicament being suitable for administration by aerial, oral, parenteral, local, intramuscular or subcutaneous routes.
